(19) United States
(12) Patent Application Publication
(10) Pub. No.: US 2018/0353533 A1
(43) Pub. Date: Dec. 13, 2018

(54) POLYINOSINIC-POLYCYTIDYLIC ACID (POLY (I:C)) PEA STARCH FORMULATION FOR THE PREVENTION AND/OR TREATMENT OF UPPER RESPIRATORY TRACT INFECTIONS

(71) Applicant: Janssen Sciences Ireland UC, Cork (IE)

(72) Inventors: Alex Henri Van Dijck, Lommel (BE); Jurgen Mensch, Laakdal (BE)

(73) Assignee: Janssen Sciences Ireland UC, Cork (IE)

(21) Appl. No.: 15/573,227

(22) PCT Filed: May 10, 2016
(86) PCT No.: PCT/IB2016/000890

§ 371 Date: Nov. 10, 2017

(30) Foreign Application Priority Data
May 11, 2015 (EP) 15167129.4

(51) Int. Cl. A61K 31/713; A61K 9/00; A61K 9/16; A61P 31/16

(52) U.S. Cl. CPC A61K 31/713; A61P 31/16; A61K 9/1652; A61K 9/0043

(57) ABSTRACT

The present invention concerns a composition comprising micro particles of polyinosinic-polycytidylic acid (Poly(I:C)) and a pea starch for us in preventing and/or treating viral infections of the upper respiratory tract such as viral respiratory infections or the common cold and a device, preferably a nasal delivery system, comprising said composition for use by a patient in need to prevent and/or treat said infections or the common cold.

### PRIORITY

This application claims priority to European Patent Application No. 15167129.4, filed May 11, 2015, which is hereby incorporated by reference in its entirety.

### BACKGROUND

The common cold (also known as nasopharyngitis, acute viral rhinopharyngitis, acute coryza, or a cold) is a viral infectious disease of the upper respiratory system caused primarily by viruses. Also influenza or influenza-like illnesses are caused by viral infections.

The common cold is a viral infection of the upper respiratory tract. The most commonly implicated virus is the rhinovirus (30-50%), a type of picornavirus with 99 known serotypes. Others include coronavirus (10-15%), influenza (5-15%), human parainfluenza viruses, human respiratory syncytial virus, adenoviruses, enteroviruses, and metapneumovirus.

In total over 200 serologically different viral types cause colds. Coronaviruses are particularly implicated in adult colds. Of over 30 coronaviruses, 3 or 4 cause infections in humans, but they are difficult to grow in the laboratory and their significance is thus less well-understood. Due to the many different types of viruses and their tendency for continuous mutation, it is impossible to gain complete immunity to the common cold.

The first indication of an upper respiratory virus is often a sore or scratchy throat. Other common symptoms are runny nose, congestion, and sneezing. These are sometimes accompanied by conjunctivitis (pink eye), muscle aches, fatigue, malaise, headache, weakness, or loss of appetite. Cough and fever generally indicate influenza rather than an upper respiratory virus with a positive predictive value of around 80%. Symptoms may be more severe in infants and young children, and in these cases it may include fever and hives. Upper respiratory viruses may also be more severe in smokers.

Viral replication begins 2 to 6 hours after initial contact. Symptoms usually begin 2 to 5 days after initial infection but occasionally occur in as little as 10 hours. Symptoms peak 2-3 days after symptom onset, whereas influenza symptom is constant and immediate. There is currently no known treatment that shortens the duration; however, symptoms usually resolve spontaneously in 7 to 10 days, with some symptoms possibly lasting for up to three weeks. In children the cough lasts for more than 10 days in 35-40% and continues for more than 25 days in 10% of the cases. The common cold is the most frequent infectious disease in humans with the average adult contracting two to four infections a year and the average child contracting several infections per year between 6-12 years of age.

The common cold is most infectious during the first two to three days of symptoms however it is also infectious for a couple of days before the onset of symptoms and may still be somewhat infectious until symptoms have completely resolved.

Human rhinovirus (HRV) is a member of the Enterovirus genus in the Picornaviridae family. The HRV particle is comprised of a 27-30 nm non-enveloped capsid consisting of 4 polypeptides (VP1, VP2, VP3, and VP4). The virus capsid contains a single-stranded RNA genome of approximately 7200 bases. A virally-encoded protein (VPg) is covalently attached to the 5′ end of the RNA genome. The clinical course of infection with human rhinovirus (HRV) has been well characterized. HRVs can infect the upper and lower airways, nasal mucosa, sinuses and middle ear, and infections produce symptoms of “the common cold” (see above). Infections are self-limiting and are typically restricted to the upper airways. Peripheral white blood cell counts may be elevated during the first 2-3 days of the infection.

HRV infection can also lead to infection of the lower airways, otitis media (particularly in young children), and sinusitis. Serious complications (such as pneumonia) from rhinovirus infection are rare and have been reported to occur in infants and young children, particularly those with underlying conditions such as bronchopulmonary dysplasia, congenital heart disease, prematurity, and neurologic conditions, and immunosuppressed (bone marrow transplant recipients) adults. While other members of the Picornaviridae family can infect the central nervous system (i.e., poliovirus, enterovirus), infection of the human central nervous system by HRVs has not been reported.

There are no commercial antiviral agents for the treatment of rhinovirus infections or prevention of common colds. Treatment of upper respiratory tract infections caused by rhinoviruses are based upon management of the symptoms (sneezing, nasal congestion, rhinorrhea, eye irritation, sore throat, cough, headaches, fever, chills) typically using over the counter antihistamines, aspirin, cough suppressants, and nasal decongestants. More serious complications of HRVs infection (e.g., pneumonia) are managed using medically appropriate standards of care.

Airway epithelial cells are the primary target of upper respiratory tract (URT) infective agents like rhino- and corona viruses. As infection with these viruses occurs prior to the onset of symptoms that reflects immune system clearance of infected cells, direct antiviral therapeutic intervention is unlikely to prove very effective. In addition, realizing and sustaining active levels of direct anti-viral compounds in the nasal mucosa is very difficult due to its high turnover. Prophylaxis on the other hand, by exploiting the body's own defenses and inducing an anti-viral state in the nasal epithelial cells, has already been shown to result in significant protection against a subsequent viral challenge as well as to lower the disease-related symptoms.

Although colds may last only a week or two, severe colds can last for up to a month. Adults average two to three colds per year and children six to ten, depending on their age and exposure. There are hundreds of different serotypes of the cold virus, making it impossible to develop a standard vaccine prophylaxis that would be effective against all of them.

Symptomatic treatment generally involves using sleep-inducing oral anti-histamines and/or vaso-constrictive decongestants that have stimulant side-effects. This is only marginally effective and these side-effects are often as debilitating as the infection itself. Although prevention would be the ideal solution, for the reasons cited above the chances of a broadly effective vaccine against all the different serotypes is highly unlikely in the near future. So, short of quarantine, people will be exposed to these infectious agents on a regular basis, especially during “cold season” and so a broadly effective, convenient, side-effect free prophylactic would have a major impact on public health and productivity in the work place.

### SUMMARY

The present invention relates to a composition comprising micro particles of polyinosinic-polycytidylic acid (Poly (I:C)) and a pea starch polymer for use in treating and/or preventing infections such as viral respiratory infections or the common cold and a device, preferably a nasal delivery system, comprising said composition for use by a patient in need to prevent and/or treat said infections or the common cold.

### BRIEF DESCRIPTION OF THE DRAWINGS

FIG. 1 consists of two panels, labeled panel (A) and panel (B). Each panel is a graph depicting the radiance of a region of interest (ROI) for mice administered microparticles comprising polyinosinic-polycytidylic acid (PolyIC) and pea starch (Lycoat RS780®) at a ratio of 1:3 (PolyIC/Lycoat RS780 1/3), 1:5 (PolyIC/Lycoat RS780 1/5), or 1:12 (PolyIC/Lycoat RS780 1/12), as well as a negative control (Placebo starch Lycoat RS780). The mice comprised a firefly luciferase gene in the interferon beta (INF-β) locus, and thus, radiance correlates with INF-β expression. Panel A and B depict the same data plotted with either a linear y-axis (A) or a logarithmic y-axis (B).

FIG. 2 consists of two panels, labeled panel (A) and panel (B). Each panel contains images of mice comprising a gene for firefly luciferase in the interferon beta (INF-β) locus that were administered luciferin, either 2-3 hours prior to administering microparticles comprising polyinosinic-polycytidylic acid (PolyIC) and pea starch (Lycoat RS780®) (panel (A)) or 24 hours after administering microparticles (panel (B)). The top row of four images in panels (A) and (B) are replicates of mice that were administered microparticles comprising polyinosinic-polycytidylic acid (PolyIC) and pea starch (Lycoat RS780®) at a ratio of 1:3 (left mouse in each image), 1:5 (center mouse in each image), or 1:12 (right mouse in each image). The middle row of four images depicts two control mice, which were each imaged separately (left image and right-center image), and two images of three mice each (left-center image and right image), which were ordered as in the top row. The bottom row of three images consists of mice that were administered microparticles comprising polyinosinic-polycytidylic acid (PolyIC) and pea starch (Lycoat RS780®) at a ratio of 1:3 (left image and center image, each depicting three mice) and mice that were administered microparticles comprising solely pea starch (Lycoat RS780®; right image, depicting three mice).

FIG. 3 consists of two panels, labeled panel (A) and panel (B). Each panel is a graph depicting the radiance of a region of interest (ROI) for mice administered microparticles comprising polyinosinic-polycytidylic acid (PolyIC) and pea starch (Lycoat RS780®) at a ratio of 1:3 (PolyIC/Lycoat RS780 1/3), 1:5 (PolyIC/Lycoat RS780 1/5), or 1:12 (PolyIC/Lycoat RS780 1/12), as well as a negative control (Placebo starch Lycoat RS780). The mice comprised a firefly luciferase gene in the interferon beta (INF-β) locus, and thus, radiance correlates with INF-β expression. Panel A and B depict the same data plotted with either a linear y-axis (A) or a logarithmic y-axis (B). FIG. 3 is based on a second experiment designed to determine whether the results of FIG. 1 are reproducible.

FIG. 4 consists of two panels, labeled panel (A) and panel (B). Each panel is a graph depicting the radiance of a region of interest (ROI) for mice administered microparticles comprising polyinosinic-polycytidylic acid (PolyIC) and pea starch (Lycoat RS780®) at a ratio of 1:3 (PolyIC/Lycoat RS780 1/3), 1:5 (PolyIC/Lycoat RS780 1/5), or 1:12 (PolyIC/Lycoat RS780 1/12), as well as a negative control (Placebo starch Lycoat RS780). The mice comprised a firefly luciferase gene in the interferon beta (INF-β) locus, and thus, radiance correlates with INF-β expression. Panel A and B depict the same data plotted with either a linear y-axis (A) or a logarithmic y-axis (B). FIG. 4 is based on pooled data, including the data points in FIGS. 1 and 3 as well as a third experiment.

### DETAILED DESCRIPTION

Various aspects of the invention relate to a formulation of a triggering molecule (Poly (I:C)) that can be used in a measurable and controllable fashion, for example, every couple of days, once a week or even every few weeks to prime the innate immune system and provide protection against viral infection. The approach outlined below takes an existing agent, Poly (I:C), which has demonstrated efficacy, but which is impractical and renders it convenient and effective using formulation sciences.

Toll-like receptor 3 (TLR3) is a protein that in humans is encoded by the TLR3 gene. TLR3 is a member of the Toll-like receptor family of pattern recognition receptors of the innate immune system which plays a fundamental role in pathogen recognition and activation of innate immunity. TLRs are highly conserved from Drosophila to humans and share structural and functional similarities. They recognize pathogen-associated molecular patterns (PAMPs) that are expressed on infectious agents, and mediate the production of cytokines necessary for the development of effective immunity. The various TLRs exhibit different patterns of expression. This TLR3 receptor is also expressed by airway epithelial cells and is restricted to the dendritic subpopulation of the leukocytes.

TLR3 recognizes double-stranded RNA (dsRNA). Double-stranded RNA is RNA with two complementary strands that can be formed during the viral replication cycle. Upon recognition, TLR 3 induces the activation of transcription factors like NF-κB and Interferon Regulatory Factor 3 (IRF3) to increase production of type I interferon which signal other cells to increase their antiviral defenses.

The structure of TLR3 forms a large horseshoe shape that contacts with a neighboring horseshoe, forming a “dimer” of two horseshoes. Much of the TLR3 protein surface is covered with sugar molecules, making it a glycoprotein, but on one face (including the proposed interface between the two horseshoes), there is a large sugar-free surface. This surface also contains two distinct patches rich in positively-charged amino acids, which may be a binding site for negatively-charged double-stranded RNA.

Polyinosine-polycytidylic acid (Poly (I:C)) is a double stranded RNA molecule with a MW distribution up to, for instance 3.600.000 Daltons. Poly (I:C) is a Toll Like Receptor 3 (TLR3) ligand that mimics viral RNA and is a known stimulant of the innate immune response. When administered nasally, it induces expression of anti-viral proteins like interferon α and β in the nasal epithelium. It has been demonstrated to reduce the number and severity of rhinovirus infections.

Poly (I:C) is usually an unstable molecule in normal aqueous solutions. Currently, to achieve an effective therapeutic or prophylactic effect, Poly (I:C) needs to be re-dissolved immediately prior to use and administered every 2 hours. To improve patient compliance and reduce the frequency of dosing, a novel formulation has been developed that is stable and shows enhanced efficacy.

Poly (I:C) has now been formulated in the present invention with a bio-adhesive polymer, pea starch, that can prolong the residence time on the nasal epithelium and provide a more effective and controllable stimulation of the innate immune system.

The current invention surprisingly provides the identification of a unique formulation that could be stored almost indefinitely at room temperature and which retains its innate immune system-stimulating activity.

The current inventive formulation contains water-soluble pea starch and has the advantage of low viscosity characteristics.

When pea starch is used in the formulation of the composition comprising Poly (I:C), such composition shows surprisingly a less sticky behavior to the inside of a vial, tube or device (like sprays) when such vials, tubes or devices must be filled with the inventive composition compared to any other starch used for the same purpose. The technical advantage is that more precisely the dosage of said Poly (I:C) can be administered to the patient in need, since less composition will stick to the inner side of the nasal spray device accordingly.

In addition the formulation enhances the efficacy of Poly (I:C) and permits much less frequent dosing with even greater TLR3 stimulating activity.

The invention therefore relates to a composition comprising micro particles of polyinosinic-polycytidylic acid (Poly (I:C)) and a pea starch polymer in the ratio Poly (I:C) over pea starch of 1:3.

Micro particles obtained and used in the current invention are particles with an average particle size between 0.1 μm and 100 μm. Preferably the carrier polymer is starch obtained from the plant genus Lathyrus, more specifically from peas.

Poly (I:C)-carrier-polymer microspheres, or also so-called micro particles, comprised in the composition are produced by means of a particle formation process such as a spray-dry process.

The Dᵥ50 (=volume based 50% cumulative undersize of the particle) of the micro particle in the composition according to the invention ranges from 0.1 μm to 200 μm, preferably from 1 μm to 50 μm, more preferably from 2 μm to 40 μm, even more preferably from 2 μm to 20 μm, and most preferred from 10 μm to 20 μm.

The Dᵥ50 (=volume based 50% cumulative undersize of the particle) of the micro particle in the composition according to the invention of 13, 14 or 15 μm is highly preferable to obtain the best performance.

The composition of the invention can be a bi-phasic suspension or a liquid composition comprising an organic solvent, wherein the organic solvent is based on glycerol or ethanol or a combination thereof. Other organic solvents serving the same purpose are trifluranes or other etherous propellants.

The composition of the invention can be used in human and/or animal medicine preferably for use in preventing and/or treating viral infections of the human upper respiratory tract such as what are referred to as “common colds.”

For animal use, the composition according to the invention can be used as aerosol formulation in for instance stables, barns, chicken flocks, and the like.

The current composition can be used by patients suffering from asthma and/or COPD (Chronic Obstructive Pulmonary Disease) in order to potentially prevent and/or treat upcoming common cold symptoms or illnesses and thus prevent exacerbations of their underlying illnesses/symptoms.

A preferred way to prevent and/or treat upper respiratory infections is performed by nasal administration of the inventive composition.

The composition of the current invention comprising micro particles of polyinosinic-polycytidylic acid (Poly (I:C)) and pea starch polymer can be used for the treatment and/or prevention of (viral) infections or common cold, wherein the composition is administered by nasal application at a time interval that is in the range of one day to one month, more preferably from every couple of days or even once a week or bi-weekly.

The above mentioned composition wherein the ratio Poly (I:C)/pea starch is 1/3 in combination with the micro particle size in the composition ranging from 0.1 μm to 200 μm, preferably from 1 μm to 50 μm, more preferably from 2 μm to 40 μm, even more preferably from 2 μm to 20 μm, and most preferred from 10 μm to 20 μm, highly preferable is 13, 14 or 15 μm, can be used for the treatment and/or prevention of (viral) infections or common cold, wherein said composition is administered by nasal application at a time interval that is in the range of one day to one month, more preferably from every couple of days or even once a week or bi-weekly.

Part of the invention is also a device, in particular a nasal delivery system, comprising a composition according to the invention.

According to the invention, Poly (I:C) is formulated as a dry powder for nasal administration. To improve stability, Poly (I:C) is spray dried from an aqueous mixture containing pea starch and Poly (I:C).

Starch is believed to have a dual function: (1) to act as a bio-adhesive in the nose, (2) and to serve as protective matrix for stabilizing Poly (I:C). Starch, especially pea starch, is a preferred excipient for nasal application as accumulation is prevented by degradation through amylases.

Starches with high amylopectin content or with chemically modified starches exhibit good muco-adhesion. Specifically hydroxypropylated (pregelatinized) pea starch (chemically modified) is used in the present invention, as these are cold water-swelling and contain a cold water-soluble fraction, resulting in a homogeneous dispersion when mixed at low shear with Poly (I:C). The resulting starch dispersions have a low to medium viscosity which allows spray drying into a homogeneous powder even at high starch ratios.

Nasal administration is preferably achieved using a single dose nasal powder device (Unit dose device supplied from Aptar Pharma Germany). The unit dose device is an active delivery system, meaning that the patient does not need to inhale and performance is patient independent. Dosing is performed by actuation, which is controlled by overpressure. The dose per puff is determined by the concentration of Poly (I:C) in the spray dried powder and the emitted weight of the powder. The powder will be administered into each nostril using a new device for each puff.

As mentioned above Poly (I:C), is a synthetic double-stranded RNA composed of anti-parallel polynucleotide strands of inosinic acid and cytidylic acid sodium salts. The strands are non-covalently bound by hydrogen bonds formed between the inosine and cytosine bases.

The average chain length for the Poly (I:C) ranges between 300 to 6,000 base pairs, corresponding to approximately 180,000 to about 3,600,000 daltons. The molecular formula is (C₁₀H₁₀N₄NaO₇P)ₓ.(C₉H₁₁NaN₃O₇P)ₓ.

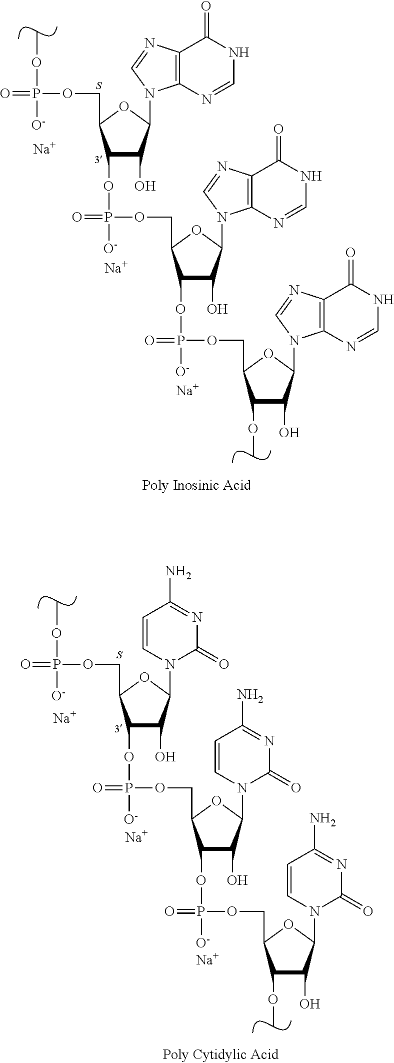

Poly (I:C) can be purchased, but it can optionally also be made in house using, for instance, the following procedure. The duplex product Poly (I:C) is manufactured from the individual homopolymers Poly Inosine (I) and Poly Cytidine (C). Poly I and Poly C are synthesized by individually polymerizing the nucleoside diphosphates inosine and cytidine in the presence of polynucleotide phosphorylase (PNPase). Each nucleoside diphosphate is individually polymerized by PNPase for 20-24 hours to control the length of the resulting ribonucleic acid polymer. The enzyme, protein kinase, is then added to terminate the polymerization reaction. The resulting homopolymers (i.e., single stranded RNA molecules) are hydrolyzed to control the molecular weight range of each polymer product within a specified range. The hydrolyzed product is treated with ethanol to precipitate the single stranded RNA molecules (ssRNA) from solution. The precipitate is separated from the supernatant and dissolved in water. The solution of ssRNA is then filtered to remove particulates, ultra filtered to remove the low-molecular weight contaminants, and then lyophilized. Lyophilized ssRNA products are individually tested for purity, molecular weight, and other quality attributes to ensure the products are within specification.

The individual single stranded homo-polymers (Poly I and Poly C) are individually dissolved in 0.015 M sodium chloride and then combined to anneal the strands forming the double stranded duplex product (Poly I:Poly C). After mixing, the resulting solution is filtered. The filtrate is ultra-filtered to remove low molecular weight contaminants. The ultra-filtered product is then lyophilized. The resulting duplex product is stored at −20° C. The lyophilized dsRNA product is tested for purity, molecular weight, and other quality attributes to ensure the product is within specification.

In some aspects, the invention relates to a composition comprising micro particles of polyinosinic-polycytidylic acid (Poly (I:C)) and a pea starch in a ratio 1/3 (w/w). The Poly (I:C)—pea starch micro particles may be produced, for example, by means of a particle formation process such as a spray-dry process. The D_{V}50 of the micro particle may range from 0.1 μm to 200 μm. The composition may be a liquid composition comprising an organic solvent, e.g., based on glycerol or ethanol or a combination thereof. In some embodiments, the invention provides a device comprising the composition, such as a nasal delivery device. The composition may be used in medicine, e.g., in treating and/or preventing infections, common cold or influenza-like illnesses. The composition may be used for the manufacture of a medicament for preventing and/or treating upper respiratory infections by nasal administration.

In some aspects, the invention relates to a microparticle, comprising polyinosinic-polycytidylic acid and a starch.

In preferred embodiments, the microparticle comprises polyinosinic-polycytidylic acid and starch at a ratio greater than 1:9. For example, the microparticle may comprise polyinosinic-polycytidylic acid and starch at a ratio of about 1:1 to about 1:8, such as about 1:1 to about 1:7, about 1:1 to about 1:6, about 1:1 to about 1:5, about 1:1 to about 1:4, about 1:1 to about 1:3, about 1:2 to about 1:8, about 1:2 to about 1:7, about 1:2 to about 1:6, about 1:2 to about 1:5, about 1:2 to about 1:4, about 1:2 to about 1:3, about 1:3 to about 1:8, about 1:3 to about 1:7, about 1:3 to about 1:6, about 1:3 to about 1:5, or about 1:3 to about 1:4. In preferred embodiments, the microparticle comprises polyinosinic-polycytidylic acid and starch at a ratio of about 1:1 to about 1:7. In more preferable embodiments, the microparticle comprises polyinosinic-polycytidylic acid and starch at a ratio of about 1:1 to about 1:6. In even more preferable embodiments, the microparticle comprises polyinosinic-polycytidylic acid and starch at a ratio of about 1:2 to about 1:5. In the most preferable embodiments, the microparticle comprises polyinosinic-polycytidylic acid and starch at a ratio of about 1:3 to about 1:5.

The microparticle may comprise polyinosinic-polycytidylic acid and starch at a ratio of about 1:1, about 1:2, about 1:3, about 1:4, about 1:5, about 1:6, about 1:7, or about 1:8. In preferred embodiments, the microparticle comprises polyinosinic-polycytidylic acid and starch at a ratio of about 1:1, about 1:2, about 1:3, about 1:4, about 1:5, about 1:6, or about 1:7. In more preferable embodiments, the microparticle comprises polyinosinic-polycytidylic acid and starch at a ratio of about 1:1, about 1:2, about 1:3, about 1:4, about 1:5, or about 1:6. In even more preferable embodiments, the microparticle comprises polyinosinic-polycytidylic acid and starch at a ratio of about 1:2, about 1:3, about 1:4, or about 1:5. In the most preferable embodiments, the microparticle comprises polyinosinic-polycytidylic acid and starch at a ratio of about 1:3, about 1:4, or about 1:5.

The starch may be maize starch (i.e., cornstarch), wheat starch, potato starch, or pea starch. The starch may be banana starch, rice starch, barley starch, rye starch, millet starch, oat starch, yam starch, sweet potato starch, cassava starch (i.e., tapioca starch), sago starch, arrowroot starch, fava bean starch, lentil starch, mung bean starch, or chickpea starch. The starch may comprise starch from more than one source, e.g., the starch may comprise pea starch, potato starch, wheat starch, and/or maize starch. Each of the above starches and combinations thereof are interchangeable with pea starch in each embodiment of the invention, and pea starch may be substituted with starch of any origin (or combinations of starches) in any embodiment of the invention. In preferred embodiments, the starch comprises a pea starch, or even consists or consists essentially of a pea starch. In some preferred embodiments, the starch comprises a maize starch (e.g., waxy maize starch), or even consists essentially of a maize starch (e.g., waxy maize starch).

The starch may comprise at least about 20% amylose, such as at least about 25% amylose or at least about 30% amylose. In certain embodiments, the starch comprises at least about 25% amylose. The starch may comprise about 20% amylose to about 85% amylose, such as about 20% to about 40%, about 30% to about 50%, about 40% to about 60%, about 50% to about 70%, about 60% to about 80%, about 20% to about 30%, about 25% to about 35%, about 30% to about 40%, about 35% to about 45%, about 40% to about 50%, about 45% to about 55%, about 50% to about 60%, about 55% to about 65%, about 60% to about 70%, about 65% to about 75%, about 70% to about 80%, or about 75% to about 85% amylose. The starch may comprise 0% to about 10% amylose, such as 0% to about 5%, 0% to about 4%, 0% to about 3%, 0% to about 2%, or 0% to about 1% amylose. The starch may comprise a high amylose starch, such as high amylose maize starch (e.g., EURYLON®). In some preferred embodiments, the starch comprises about 15% amylose to about 50% amylose, more preferably about 20% amylose to about 45% amylose, and most preferably about 25% amylose to about 40% amylose. In other preferred embodiments, the starch may comprise less than about 5% amylose, such as less than 4%, less than about 3%, less than about 2%, or less than about 1% amylose. The fraction of the starch that is not amylose is preferably amylopectin, e.g., a starch that comprises about 20% to about 40% amylose preferably comprises about 60% to about 80% amylopectin and a starch that comprises 0% to about 10% amylose preferably comprises about 90% to 100% amylopectin.

The starch may comprise about 15% to about 80% amylopectin, such as about 20% to about 40%, about 30% to about 50%, about 40% to about 60%, about 50% to about 70%, about 60% to about 80%, about 15% to about 25%, about 20% to about 30%, about 25% to about 35%, about 30% to about 40%, about 35% to about 45%, about 40% to about 50%, about 45% to about 55%, about 50% to about 60%, about 55% to about 65%, about 60% to about 70%, about 65% to about 75%, or about 70% to about 80% amylopectin. The starch may comprise about 90% to 100% amylopectin, such as about 95% to 100%, about 96% to 100%, about 9′7% to 100%, or about 98% to 100% amylopectin. In some preferred embodiments, the starch comprises about 50% to about 85% amylopectin, more preferably about 55% to about 80% amylopectin, and most preferably about 60% to about 75% amylopectin. In some preferred embodiments, the starch comprises at least about 90% amylopectin, such as at least about 95%, about 96%, about 97%, about 98%, or at least about 99% amylopectin. The fraction of the starch that is not amylopectin is preferably amylose, e.g., a starch that comprises about 60% to about 70% amylopectin preferably comprises about 30% to about 40% amylose and a starch that comprises about 95% to 100% amylopectin preferably comprises 0% to about 5% amylose.

In some preferred embodiments, the starch is a pregelatinized starch. For example, in certain, preferred embodiments, the starch may be pregelatinized pea starch or pregelatinized maize starch (e.g., pregelatinized waxy maize starch). Starch gelatinization may increase the solubility of the starch.

The starch may be a modified starch, such as dextrin, acid-treated starch, alkaline-treated starch, bleached starch, oxidized starch, enzyme-treated starch, monostarch phosphate, distarch phosphate, phosphated distarch phosphate, acetylated distarch phosphate, starch acetate, acetylated distarch adipate, hydroxypropyl starch, hydroxypropyl distarch phosphate, hydroxypropyl distarch glycerol, starch sodium octenyl succinate, starch aluminum octenyl succinate, acetylated oxidized starch, cationic starch, hydroxyethyl starch, or carboxymethylated starch. Modification may alter the hydrophobicity, hydrophilicity, charge, hygroscopicity, viscosity, and/or solubility of the starch. Lycoat RS780®, for example, is a pregelatinized hydroxypropyl pea starch, wherein some of the hydroxyl groups of the pea starch have been substituted with a hydroxypropyl group. In some embodiments, 0% to about 40% of the hydroxyl groups of the starch are hydroxypropylated, such as 1% to about 20%, or about 2% to about 10%. In some embodiments, 0% to about 40% of the hydroxyl groups of the starch are hydroxyethylated, such as 1% to about 20%, or about 2% to about 10%. In certain preferred embodiments, the starch is a hydroxypropyl starch, such as a hydroxypropyl pea starch. In certain preferred embodiments, the starch is a pregelatinized hydroxypropyl starch, such as a pregelatinized hydroxypropyl pea starch. In certain preferred embodiments, the starch is a hydroxyethyl starch, such as hydroxyethyl maize starch. In certain preferred embodiments, the starch is a pregelatinized hydroxyethyl starch, such as a pregelatinized hydroxyethyl maize starch.

In some embodiments, the average chain length of the polyinosinic-polycytidylic acid is approximately 300 base pairs to 6,000 base pairs. For example, the polyinosinic-polycytidylic acid may comprise polyinosinic acid having an average chain length of approximately 300 nucleotides to 6,000 nucleotides and polyinosinic acid having an average chain length of approximately 300 nucleotides to 6,000 nucleotides. In some embodiments, the average molecular weight of the polyinosinic-polycytidylic acid is approximately 180 kDa to 3,600 kDa. The polyinosinic-polycytidylic acid may be present as a salt, such as a salt of sodium.

In some embodiments, the microparticle may comprise water. A microparticle may consist essentially of polyinosinic-polycytidylic acid, starch, and water, e.g., wherein polyinosinic-polycytidylic acid comprises sodium or another cation. A microparticle may consist of polyinosinic-polycytidylic acid, starch, and water.

The microparticle may have a size from 0.1 μm to 200 preferably from 1 μm to 50 μm, more preferably from 2 μm to 40 even more preferably from 2 μm to 20 μm, and most preferred from 10 μm to 20 μm. The microparticle may have a size of about 2 μm to about 30 μm, such as about 4 μm to about 30 μm, about 5 μm to about 30 μm, or about 6 μm to about 30 μm. The microparticle may have a size of about 2 μm to about 27 μm, such as about 4 μm to about 27 μm, about 5 μm to about 27 μm, or about 6 μm to about 27 μm. The microparticle may have a size of about 2 μm to about 20 μm, such as about 4 μm to about 20 μm, about 5 μm to about 20 μm, or about 6 μm to about 20 μm. The microparticle may have a size of about 2 μm to about 10 μm, such as about 4 μm to about 10 μm, about 5 μm to about 10 μm, or about 6 μm to about 10 μm.

In some aspects, the invention relates to a composition comprising a plurality of microparticles as described herein (e.g., supra). The composition may have a Dᵥ50 of about 1 μm to about 200 μm, such as about 1 μm to about 50 μm, about 2 μm to about 40 μm, about 2 μm to about 20 μm, or about 10 μm to about 20 μm. The composition may have a Dᵥ50 of about 4 μm to about 20 μm, about 5 μm to about 20 μm, about 6 μm to about 20 μm, about 7 μm to about 20 μm, about 8 μm to about 20 μm, about 9 μm to about 20 μm, or about 10 μm to about 20 μm.

A composition comprising a plurality of microparticles may be a liquid or a dry powder. A composition comprising a plurality of microparticles may comprise an organic solvent such as glycerol, ethanol, or a combination thereof. A composition comprising a plurality of microparticles may further comprise phosphate-buffered saline. In preferred embodiments, a composition comprising a plurality of microparticles is stable during storage at room temperature for at least 1 month, such as at least 2 months, at least 3 months, at least 4 months, at least 5 months, at least 6 months, at least 7 months, at least 8 months, at least 9 months, at least 10 months, at least 11 months, or at least 12 months. A composition comprising a plurality of microparticles may be stable during storage at room temperature for about 1 month, about 2 months, about 3 months, about 4 months, about 5 months, about 6 months, about 7 months, about 8 months, about 9 months, about 10 months, about 11 months, or about 12 months.

In some aspects, the invention relates to a nasal delivery system, comprising a composition comprising microparticles as described herein (e.g., supra).

In some aspects, the invention relates to the use of a composition comprising a plurality of microparticles as described herein for the manufacture of a medicament for prophylacting against and/or treating upper respiratory infections. In some embodiments, the invention relates to the use of a composition comprising microparticles as described herein for the manufacture of a medicament for prophylacting against upper respiratory viral infections in a subject having chronic obstructive pulmonary disease (COPD).

A composition comprising a plurality of microparticles as described herein may be useful for treating or prophylacting against a viral infection of the upper respiratory tract, such as a rhinovirus infection or an influenza virus infection. The viral infection may be caused by a picornavirus (e.g., rhinovirus), coronavirus, influenza virus, human parainfluenza virus, human respiratory syncytial virus, adenovirus, enterovirus, or metapneumovirus. A composition comprising a plurality of microparticles as described herein may be useful for prophylacting against upper respiratory viral infections in a subject having chronic obstructive pulmonary disease (COPD), or a symptom thereof. A method may comprise administering the composition to a subject, e.g., by nasal administration. The subject may be a human or an animal, preferably human.

In some aspects, the invention relates to a method of treating or prophylacting against a viral infection in a subject, comprising administering to the subject a composition comprising a plurality of microparticles as described herein. The viral infection may be caused by a picornavirus (e.g., rhinovirus), coronavirus, influenza virus, human parainfluenza virus, human respiratory syncytial virus, adenovirus, enterovirus, or metapneumovirus. In certain preferred embodiments, the viral infection is a rhinovirus infection or an influenza virus infection. Administering a composition may comprise, for example, nasal administration. The subject may be a human or an animal, preferably human.

In some aspects, the invention relates to a method of prophylacting against upper respiratory viral infections in a subject having chronic obstructive pulmonary disease (COPD), comprising administering to the subject a composition comprising a plurality of microparticles as described herein. Administering a composition may comprise, for example, nasal administration. The subject may be a human or an animal, preferably human.

### EXEMPLIFICATION

### Example 1. Methods of Preparing and Characterizing Microparticles

Spray Drying of Poly (I:C) with Pea Starch

The spray dry process was performed on a Buchi B290 Mini spray dryer (Buchi, Flawil, Switzerland). Nuclease free water added to a glass beaker and the pea starch is added while mixing using a magnetic stirrer until the starch is completely dispersed. Poly (I:C) was dissolved in nuclease free water and stirred on a magnetic stirrer until the Poly (I:C) is completely dissolved. The dissolved Poly (I:C) is added to the dispersed pea starch and stirred at room temperature overnight. A total solids concentration of 4.7% (w/w) and a ratio of Poly (I:C)/pea starch 1/3 (w/w) was applied.

The solutions were fed to a two-fluid nozzle (diameter: 0.7 mm) at the top of the spray dryer by means of a peristaltic pump. The spray dryer operated in co-current nitrogen flow mode. The spray dried particles were collected in a reservoir attached to a cyclone. After collection of the particles, the glass cylinder and cyclone was cooled to room temperature. The collected powder was transferred to amber glass bottle and this bottle is placed in an aluminum vapor lock bag. The vials were stored at room temperature.

### Scanning Electron Microscopy

The samples were sputtered with gold particles with diameter +/−30-50 nm. Images were generated using a FEI scanning electron microscope-type Quanta 200F with Everhart Thornley detector.

### Water Content—Karl Fischer Titration

Water content of the concepts was determined by means of a direct volumetric Karl Fisher titration. A KF TITRATOR V30 is used (Mettler Toledo, US). The powder (50-100 mg) was transferred to the titration vessel containing Hydranal® Methanol Dry (Sigma Aldrich) and stirred for 300 seconds. Titration was performed with Hydranal® Composite 2 (Sigma Aldrich) at a concentration of 2 mg/ml using a 5 ml burette. For termination, a stop drift of 15 μg/min was applied. Samples were analyzed in triplicate.

### Determination of Particle Size

There exists a tendency to evaluate particle size distribution data merely on the basis of the volume distribution of the products of interest. Thereby, one often limits the valuation to a comparison of the Dᵥ10, Dᵥ50 and Dᵥ90 cumulative undersizes. However, comparing dᵥx cumulative undersizes may not always be straight-forward due to the fact that different techniques and instruments readily lead to different results. In addition, one can get more information out of a particle size (or shape) distribution data by looking from a different perspective to the data (i.e., using other parameters).

For the determination of the particle size distribution the laser diffraction test method was used. The analysis was performed on a Malvern Mastersizer 2000 laser diffractometer equipped with a Hydro2000S wet dispersion module (or an equivalent system). The instrument is used in the blue light ON detection mode at a size range of 20 nm to 2 mm. The measured particle size distribution by volume in the current invention for Dᵥ10 is 4 μm, for Dᵥ50 it is 14 μm while for Dᵥ90 it is 27 μm.

### Example 2. In Vivo Testing of Formulations in the Influenza Mouse Model

All animal studies were approved by the ethical committee and performed according to national and international guidelines. 8-12 week old female Swiss mice (Janvier) were used. All intranasal treatments were performed under isoflurane anesthesia. To administer an amount of liquid, a droplet was applied directly on top of the nostril and, by closing the mouth, the droplet was allowed to enter via the nostril into the nasal cavity. Spray dried Poly (I:C)—pea starch powders were freshly prepared just prior to each experiment and administrated with a powder tip device. Unformulated Poly (I:C) was administrated in phosphate buffered saline (PBS) at a concentration of 1 mg/ml.

Testing the IFN-β Inducing Capacity of polyIC/Lycoat RS780 (=Pea Starch) in Different Ratios (1/3, 1/5, 1/12)

The luciferase reporter for interferon-beta (IFN-β) gene activation can provide insight in the activation of IFN-β after stimulation with different poly IC formulations. Poly IC is a synthetic analog which mimics dsRNA viruses by stimulation of the innate immune system through Pattern Recognition Receptors (PRR). When polylC binds to its PRR (TLR3), RIG-1 and/or MDA 5, a signaling cascade is started and results in activation of type I interferons, of which IFN-β is a representative. The heterozygous IFN-β+/Δβ-luc albino (Tyrc2J) C57BL/6 mice produce firefly luciferase driven by the IFN-β promoter due to a targeted mutation in the IFN-β locus. For optical imaging luciferin is administered systemically and photon emission is monitored using an IVIS200 system (CaliperLS).

The IFN-β inducing capacity of a compound in a dry powder formulation was tested in different ratios PolyIC/Lycoat RS780 (=pea starch) 1/3, 1/5, and 1/12. A pea starch-only formulation was included as a negative control. The compounds were administered intranasally to IFN-β reporter mice and in vivo imaging was performed before and 24 h after administration.

### Experimental Protocol

### Mice.

The generation of the IFN-β reporter mice has been previously described (Lienenklaus et. al. 2009). Briefly, the mice produce firefly luciferase driven by the IFN-β promoter due to a targeted mutation in the IFN-β locus. The mice used in this study were heterozygous IFN-β+/Δβ-luc albino (Tyrc2J) C57BL/6. Males and females between the age of 12 and 14 weeks were used. The animals are housed in IVC racks and they are provided with food and water ad libitum.

### Administration of Compounds.

Thirty one 8-12 week old male and female heterozygous IFN-β+/Δβ-luc albino (Tyrc2J) C57BL/6 were used. All intranasal administrations were performed under injection anesthesia (ketamine/xylazine). The dry powder was administered using a self-made tip device. The tip-devices were used to administer 2 mm of powder into the left nostril and an additional 2 mm of powder onto the nose. After the administration of the powder, the mice were placed under a red lamp to wake up.

### Imaging of Mice.

For in vivo imaging mice were i.v. injected with D-Luciferin, firefly, potassium salt (30 mg/ml in PBS; 100 μl/20 g mouse). Mice were anesthetized with isoflurane, and photon emission was monitored using an IVIS200 system (CaliperLS) ˜5 min after injection of luciferin. Imaging was performed 4 hours before (background signal) and 24 hours after compound administration. Data correction for the time span between luciferin administration and imaging was done: Corrected flux=total flux+Δt[min]*(0.0459*total flux).

### Results

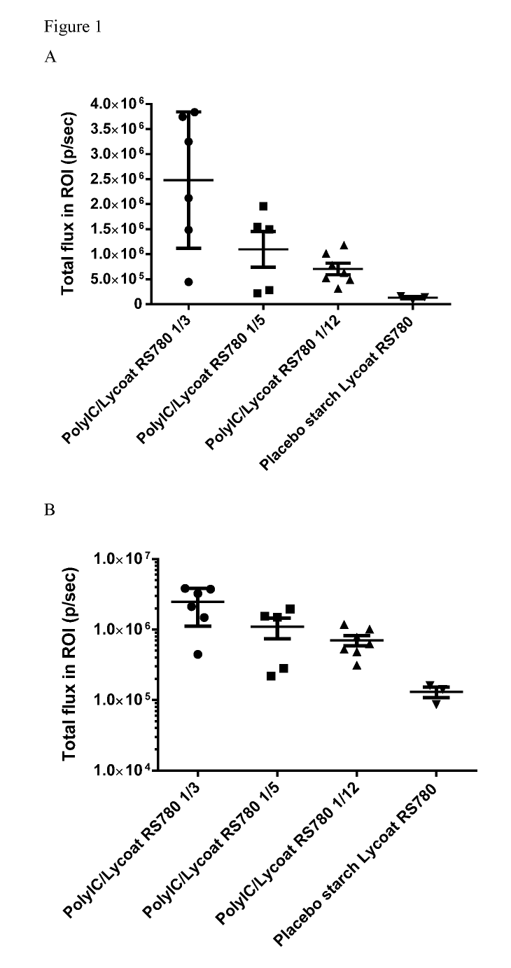

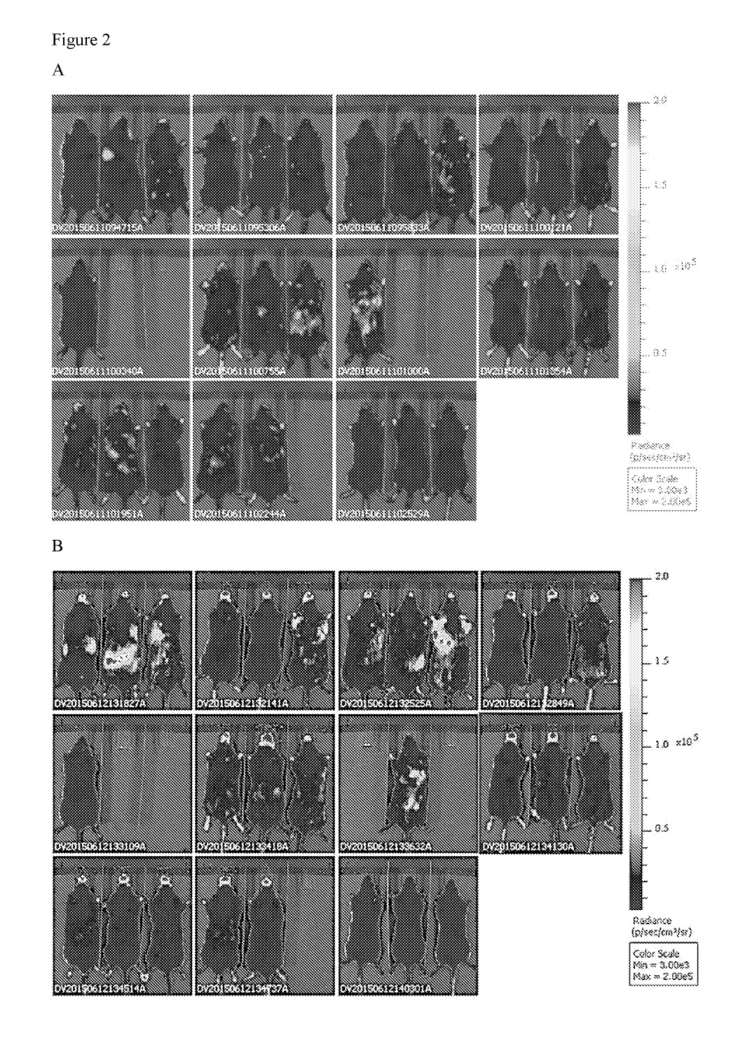

Statistical analysis of the actual data showed a significant difference between the groups PolyIC/Lycoat RS780 (=pea starch) 1/3 versus 1/12 and also between the PolyIC/Lycoat RS780 1/3 versus the Placebo starch Lycoat RS780 group (FIG. 1). The statistical analysis of the log transformed data showed a significant difference between the groups PolyIC/Lycoat RS780 1/3 versus Placebo starch Lycoat RS780 group, the 1/5 group versus the placebo group and the 1/12 group versus the placebo group (FIG. 2). In conclusion, there is a good correlation between the activity of the formulation (IFN response) and the ratio of PolyIC/Lycoat RS780 in the formulation. The IFN response increases when a higher ratio of PolyIC/Lycoat RS780 starch is used.

### Example 3. Reproducibility of In Vivo Testing

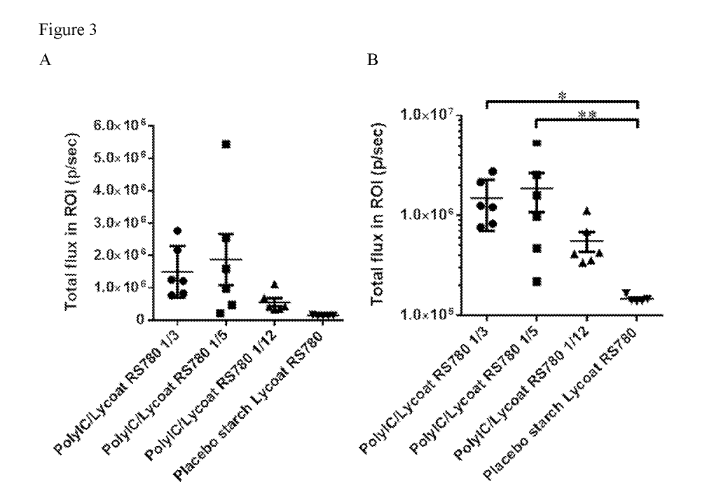

The methods of Example 2 were repeated. FIG. 2 depicts mice imaged for luciferase activity before (FIG. 2A) and after (FIG. 2B) administering the microparticles. FIG. 3 depicts the measured radiance of the mice after administering microparticles comprising either a 1:3, 1:5, or 1:12 polyinosinic-polycytidylic acid to starch (Lycoat RS780®) ratio, or the negative control (Placebo starch Lycoat RS780). No statistical significance was observed in a linear analysis of radiance (FIG. 3A), but statistical significance was observed between the 1:3 group and negative control (p=0.0002) and between the 1:5 group and the negative control (p=0.0006) after performing a logarithmic transform on the data (FIG. 3B). The statistical significance between the 1:5 group and the negative control was lost, however, upon deleting a data point associated with a high responder (p=0.1897).

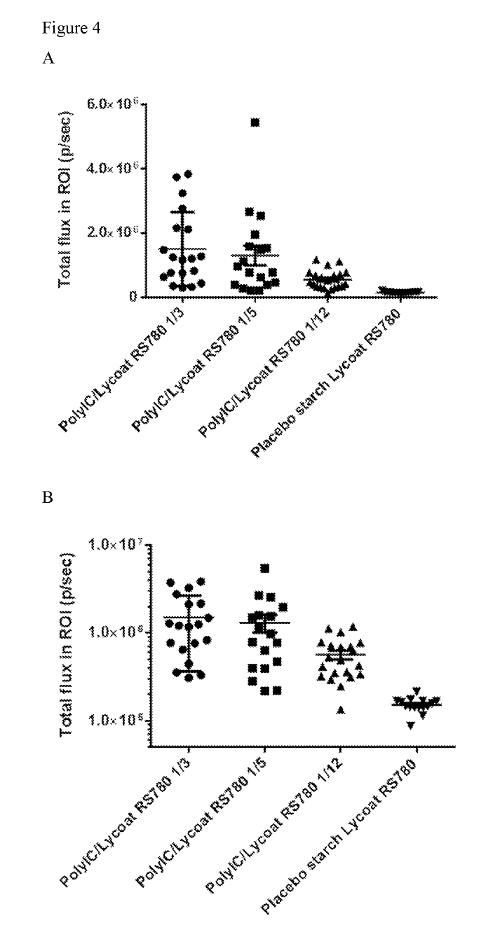

The data from Examples 2 and 3 were pooled with data from a third, similarly performed experiment (FIG. 4). When the data was analyzed using a linear analysis, statistical significance was observed between the 1:3 group and negative control (p=0.0002), between the 1:3 group and the 1:12 group (p=0.0060), and between the 1:5 group and the negative control (p=0.0020). When the data was analyzed after performing a logarithmic transform, statistical significance was observed between the 1:3 group and negative control (p<0.0001), between the 1:3 group and the 1:12 group (p=0.0017), between the 1:5 group the negative control (p<0.0001), between the 1:5 group the 1:12 group (p<0.0001), and between the 1:12 group the negative control (p<0.0001), but not between the 1:3 group and the 1:5 group (p=0.7064).

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the disclosure.

What is claimed is:

1. A microparticle, comprising polyinosinic-polycytidylic acid and a starch, wherein the ratio of polyinosinic-polycytidylic acid to starch in the microparticle is about 1:1 to about 1:5.

2. The microparticle of claim 1, wherein the ratio of polyinosinic-polycytidylic acid to starch in the microparticle is about 1:3 to about 1:5.

3. The microparticle of claim 2, wherein the ratio of polyinosinic-polycytidylic acid to starch in the microparticle is about 1:3 or about 1:5.

4. The microparticle of any one of the preceding claims, wherein the starch is a pregelatinized starch.

5. The microparticle of any one of the preceding claims, wherein the starch is a pea starch.

6. The microparticle of any one of the preceding claims, wherein the average chain length of the polyinosinic-polycytidylic acid is approximately 300 base pairs to 6,000 base pairs.

7. The microparticle of any one of the preceding claims, wherein the average molecular weight of the polyinosinic-polycytidylic acid is approximately 180 kDa to 3,600 kDa.

8. The microparticle of any one of the preceding claims, wherein the polyinosinic-polycytidylic acid is present as a sodium salt.

9. The microparticle of any one of the preceding claims, further comprising water.

10. The microparticle of any one of the preceding claims, wherein the microparticle consists essentially of polyinosinic-polycytidylic acid, starch, and water.

11. The microparticle of any one of the preceding claims, wherein the microparticle has a size between 0.1 μm and 100 μm.

12. The microparticle of claim 11, wherein the microparticle has a size from 4 μm to 27 μm.

13. The microparticle of claim 11, wherein the microparticle has a size from 2 μm to 20 μm.

14. A composition, comprising a plurality of microparticles according to any one of the preceding claims.

15. A composition comprising microparticles, wherein the microparticles comprise polyinosinic-polycytidylic acid (Poly (I:C)) and a starch.

16. The composition of claim 14 or 15, wherein the microparticles of the composition have a Dv50 from 0.1 μm to 200 μm.

17. The composition of claim 16, wherein the microparticles of the composition have a Dv50 from 1 μm to 50 μm.

18. The composition of claim 17, wherein the microparticles of the composition have a Dv50 from 2 μm to 20 μm.

19. The composition of any one of claims 14 to 18, wherein the composition is a liquid.

20. The composition of any one of claims 14 to 19, wherein the composition comprises an organic solvent.

21. The composition of claim 20, wherein the organic solvent is glycerol, ethanol, or a combination thereof.

22. The composition of any one of claims 14 to 19, wherein the composition comprises phosphate-buffered saline.

23. The composition of any one of claims 14 to 18, wherein the composition is a dry powder.

24. The composition of claim 23, wherein the particles are stable during storage at room temperature for one month.

25. The composition of any one of claims 14 to 24, for use in a method of treating or prophylacting against a viral infection of the upper respiratory tract, wherein the method comprises administering the composition to a patient.

26. The composition for use of claim 25, wherein the viral infection is a human rhinovirus infection or an influenza virus infection.

27. The composition of any one of claims 14 to 24, for use in a method of prophylacting against a viral infection of the upper respiratory tract in a patient having chronic obstructive pulmonary disease (COPD), wherein the method comprises administering the composition to a patient.

28. A composition for use in a method of treating or prophylacting against a viral infection of the upper respiratory tract, wherein:
the composition comprises microparticles;
the microparticles comprise polyinosinic-polycytidylic acid (Poly (I:C)) and starch at a ratio of about 1:1 to about 1:5; and
the method comprises administering the composition to a patient.

29. A composition for use in a method of prophylacting against a viral infection of the upper respiratory tract in a subject having chronic obstructive pulmonary disease (COPD), wherein:
the composition comprises microparticles;
the microparticles comprise polyinosinic-polycytidylic acid (Poly (I:C)) and starch at a ratio of about 1:1 to about 1:5; and
the method comprises administering the composition to a patient.

30. The composition for use of any one of claims 25 to 29, wherein administering the composition comprises nasal administration.

31. A method of treating or prophylacting against a viral infection in a subject, comprising administering to the subject the composition of any one of claims 14 to 24.

32. The method of claim 31, wherein the viral infection is a human rhinovirus infection or an influenza virus infection.

33. A method of prophylacting against a viral infection of the upper respiratory tract in a subject having chronic obstructive pulmonary disease (COPD), comprising administering to the subject the composition of any one of claims 14 to 24.

34. A method of treating or prophylacting against a viral infection in a subject, comprising administering to the subject a composition comprising microparticles, wherein the microparticles comprise polyinosinic-polycytidylic acid (Poly (I:C)) and starch at a ratio of about 1:1 to about 1:5.

35. The method of claim 34, wherein the viral infection is a human rhinovirus infection or an influenza virus infection.

36. A method of prophylacting against a viral infection of the upper respiratory tract in a subject having chronic obstructive pulmonary disease (COPD), comprising administering to the subject a composition comprising microparticles, wherein the microparticles comprise polyinosinic-polycytidylic acid (Poly (I:C)) and starch at a ratio of about 1:1 to about 1:5.

37. The method of any one of claims 31 to 36, wherein administering the composition comprises nasal administration.

38. A nasal delivery system, comprising the composition of any one of claims 14 to 30.

39. A nasal delivery system, comprising a composition comprising microparticles, wherein the microparticles comprise polyinosinic-polycytidylic acid (Poly (I:C)) and starch at a ratio of about 1:1 to about 1:5.

40. Use of the composition of any one of claims 14 to 24, for the manufacture of a medicament for prophylacting against and/or treating upper respiratory viral infections.

41. Use of a composition comprising microparticles for the manufacture of a medicament for prophylacting against and/or treating upper respiratory viral infections, wherein the microparticles comprise polyinosinic-polycytidylic acid (Poly (I:C)) and starch at a ratio of about 1:1 to about 1:5.

42. Use of the composition of any one of claims 14 to 24, for the manufacture of a medicament for prophylacting against a viral infection of the upper respiratory tract in a subject having chronic obstructive pulmonary disease (COPD).

43. Use of a composition comprising microparticles for the manufacture of a medicament for prophylacting against a viral infection of the upper respiratory tract in a subject having chronic obstructive pulmonary disease (COPD), wherein the microparticles comprise polyinosinic-polycytidylic acid (Poly (I:C)) and starch at a ratio of about 1:1 to about 1:5.
